(19) United States
(12) Patent Application Publication
(10) Pub. No.: US 2008/0194531 A1
(43) Pub. Date: Aug. 14, 2008

(54) Methods for Treating Visual Disorders

(76) Inventors: Clifford J. Steer, St. Paul, MN (US); Walter C. Low, Shorewood, MN (US); Timothy W. Olson, Eden Prairie, MN (US); Jeffrey H. Boatright, Decatur, GA (US); John M. Nickerson, Decatur, GA (US); Cecilia M.P. Rodrigues, Lisbon (PT); Machelle T. Pardue, Lawrenceville, GA (US)

Correspondence Address: MUETING, RAASCH & GEBHARDT, P.A., P.O. BOX 581336, MINNEAPOLIS, MN, 55458-1336, US

(21) Appl. No.: 11/883,977

(22) PCT Filed: Feb. 8, 2006
(86) PCT No.: PCT/US06/04394

§ 371 Date: Mar. 28, 2008

Related U.S. Application Data
(60) Provisional application No. 60/651,729, filed on Feb. 10, 2005.

(51) Int. Cl. A61K 31/56; A61P 27/00

(52) U.S. Cl. 514/182

(57) ABSTRACT

The present invention provides methods for treating visual disorders. Exemplary visual disorders include macular degeneration, retinitis pigmentosa, glaucoma, and/or retinal degeneration.

### CROSS REFERENCE TO RELATED APPLICATION

This application claims the benefit of U.S. Provisional Application Ser. No. 60/651,729, filed Feb. 10, 2005, which is incorporated herein by reference in its entirety.

### SUMMARY

The present invention provides methods for treating visual disorders. Exemplary visual disorders include macular degeneration, retinitis pigmentosa, glaucoma, and/or retinal degeneration.

In one embodiment, a method includes administering to a subject a compound selected from the group of a hydrophilic bile acid, salts thereof, analogs thereof, or combinations thereof. In one embodiments, the hydrophilic bile acid is ursodeoxycholic acid. In one embodiment, the compound administered is glycol- or tauro-ursodeoxycholic acid. In one embodiment, the compound is administered in combination with a pharmaceutically acceptable carrier.

In one embodiment, the method includes contacting an eye of a subject a compound selected from the group of a hydrophilic bile acid, salts thereof, analogs thereof, or combinations thereof, wherein the visual disorder is macular degeneration, retinitis pigmentosa, glaucoma, and/or retinal degeneration

In one embodiment, administering to a subject includes contacting the eye of the subject with a hydrophilic bile acid, salts thereof, analogs thereof, or combinations thereof.

In one embodiment, administering involves administering parenterally. In one embodiment, administering involves administering the compound in eye drops.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF ILLUSTRATIVE EMBODIMENTS

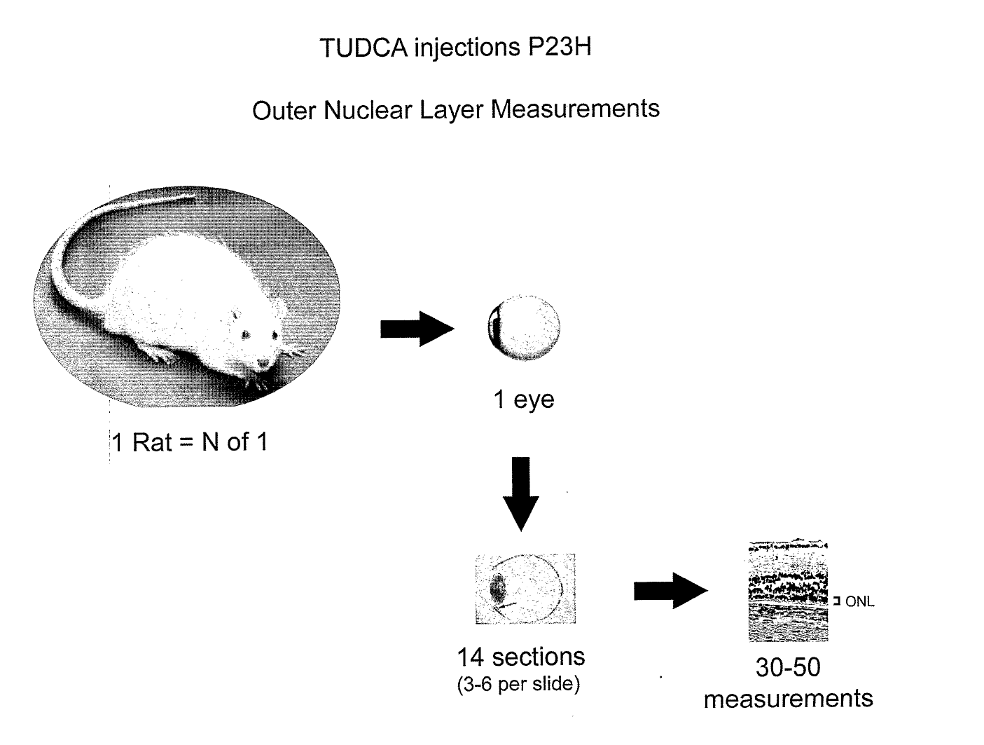

FIG. 1. Study design.

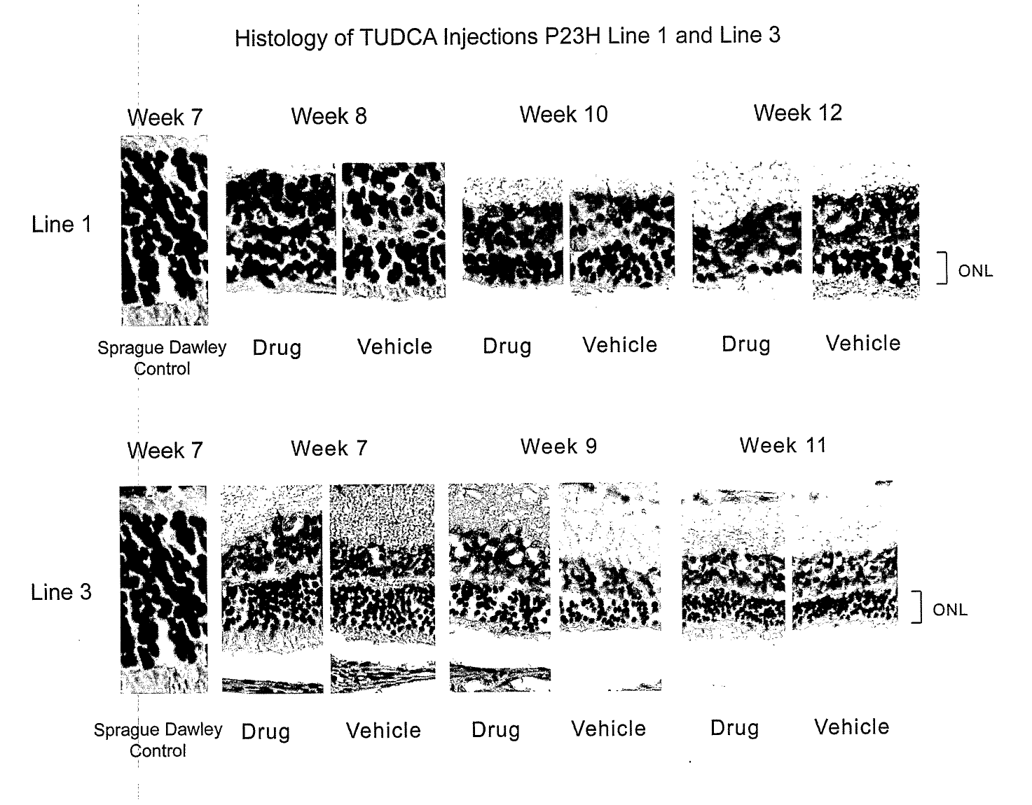

FIG. 2. A representative image of the retinal degeneration shown in sequential histophathologic images representative of the time point based on days from birth, and the influence of TUDCA.

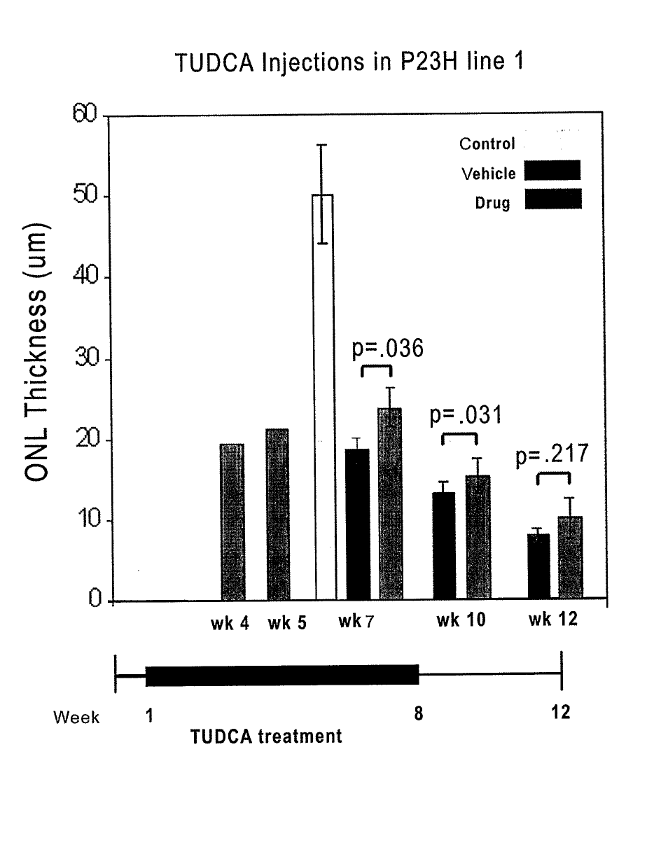

FIG. 3. Data showing animals treated the TUDCA as compared to vehicle controls (see week 7, 10, and 12).

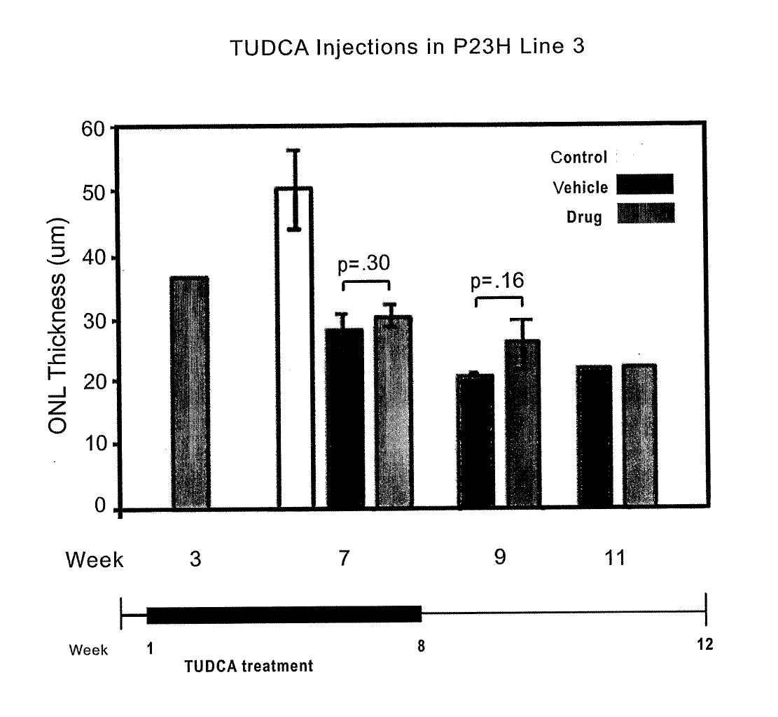

FIG. 4. Data showing a trend toward a protective effect of TUDCA on the rate of retinal degeneration.

### DETAILED DESCRIPTION OF ILLUSTRATIVE EMBODIMENTS

The present invention provides methods that involve the treatment of visual disorders, including macular degeneration, retinitis pigmentosa, glaucoma, retinal degeneration (e.g., rod photoreceptor degeneration).

The methods of the present invention involve administering to a subject (particularly, contacting the eye of a subject) with a hydrophilic bile acid, salts thereof, analogs thereof, or combinations thereof. As used herein, hydrophilic bile acids are those more hydrophilic than deoxycholic acid (DCA). This can be determined by evaluating the partition coefficient between water and octanol, with the more hydrophilic bile acids being more favorable toward water. Alternatively, the more hydrophilic bile acids have earlier retention times on a reverse-phase column using high performance liquid chromatography. A particularly preferred hydrophilic bile acid includes ursodeoxycholic acid. Examples of analogs of hydrophilic bile acids include conjugated derivatives of bile acids. Two particularly preferred conjugated derivatives include glyco- and tauro-ursodeoxycholic acid.

Although all hydrophilic bile acids may not be useful in all methods of the present invention, they can be evaluated readily by a method similar to that mentioned above. Such compounds are used in amounts effective to treat (including prevent) a visual disorder, whether it be prophylactically or therapeutically. They can be used in the methods of the present invention in the form of a composition that also includes a pharmaceutically acceptable carrier, if so desired. Typically, for preferred embodiments, the compounds described herein are formulated in pharmaceutical compositions, and then, in accordance with methods of the invention, administered to a mammal, such as a human patient, in a variety of forms adapted to the chosen route of administration. The formulations include those particularly suitable for ophthalmic administration (e.g., eye drops) or other local methods, although other modes of administration such as oral or parenteral (including subcutaneous, intramuscular, intraperitoneal and intravenous) administration may be possible. Local drug delivery methods include subtenon's, subconjunctival, intravitreal, topical, suprachoroidal, peribulbar, or from a local delivery device that utilizes the transscleral route. Treatment can be prophylactic, or alternatively, can be initiated after diagnosis of the visual disorder. That is, compounds of the present invention can be used to prevent the onset and/or progression of a visual disorder.

The formulations may be conveniently presented in unit dosage form and may be prepared by any of the methods well known in the art of pharmacy. All methods include the step of bringing the active compound into association with a carrier that constitutes one or more accessory ingredients. In general, the formulations are prepared by uniformly and intimately bringing the active compound into association with a liquid carrier, a finely divided solid carrier, or both, and then, if necessary, shaping the product into a desired formulation.

Formulations of the present invention suitable for oral administration may be presented as discrete units such as tablets, troches, capsules, lozenges, wafers, implants, or cachets, each containing a predetermined amount of the compound as a powder, in granular form, incorporated within liposomes, or as a solution or suspension in an aqueous liquid or non-aqueous liquid such as a syrup, an elixir, an emulsion, or a draught. Such compositions and preparation should contain at least about 500 mg/day to about 1000 mg/day, or alternatively stated, about 10 mg/kg body weight to about 15 mg/kg body weight.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid and the like; a lubricant such as magnesium stearate; a sweetening agent such as sucrose, fructose, lactose or aspartame; and a natural or artificial flavoring agent. When the unit dosage form is a capsule, it may further contain a liquid carrier, such as a vegetable oil, a polyethylene glycol, in poly(ortho esters), or poly(lactic-co-glycolic) acid microspheres. Various other materials may be present as coatings or to otherwise modify the physical form of the solid unit dosage form. For instance, tablets, pills, or capsules may be coated with gelatin, wax, shellac, or sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The compound may be incorporated into sustained-release preparations and devices.

Formulations suitable for parenteral administration conveniently comprise a sterile aqueous preparation of the compound, or dispersions of sterile powders comprising the compound, which are preferably isotonic with the blood of the recipient. Isotonic agents that can be included in the liquid preparation include sugars, buffers, and salts such as sodium chloride. Solutions of the compound can be prepared in water, optionally mixed with a nontoxic surfactant. Dispersions of the compound can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the compound, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the compounds over a prolonged period can be achieved by including agents for delaying, for example, aluminum monostearate and gelatin.

Eye drop formulations are preferred and comprise purified aqueous solutions of the compound with preservative agents and isotonic agents. Such formulations are preferably adjusted to a pH and isotonic state compatible with the eye.

In addition to the aforementioned ingredients, the formulations of this invention may further include one or more accessory ingredients including diluents, buffers, binders, disintegrants, surface active agents, thickeners, lubricants, preservatives (including antioxidants) and the like.

Useful dosages of the compounds described herein can be determined by comparing their in vitro activity and the in vivo activity in animal models. Methods for extrapolation of effective dosages in mice, and other animals, to humans are known in the art.

Generally, for adult humans, single dosages for injection, infusion, or ingestion will generally vary from about 500 mg to about 1000 mg (i.e., a dosage of about 10 mg to about 15 mg per kg of body weight per day). It may be administered, for example, about 1 to about 3 times per day, to yield levels of about 10 to about 15 micromoles per liter of serum.

Advantages of the invention are illustrated by the following examples. However, the particular materials and amounts thereof recited in these examples, as well as other conditions and details, are to be interpreted to apply broadly in the art and should not be construed to unduly limit the invention.

### EXAMPLES

### Treatment of Neurosensory Retinal Degenerations with Bile Acid

We have studied the use of bile acids (tauroursodeoxycholic acid, TUDCA) for inhibition of neurosensory retinal degeneration in an animal model of retinal degeneration (P23H rat).

Retinal Degenerations: Age-Related Macular Degeneration (AMD) is the leading cause of blindness in the United States and Western World in individuals over age 50. Early changes of AMD are common. In fact, by age 65, nearly 25% of individuals will demonstrate signs of early AMD, while 1-2% will have late AMD or severe vision loss (Beaver Dam Eye Study, Beaver Dam Wisconsin, R. Klein et al). Inherited retinal degeneration (such as retinitis pigmentosa) is the leading cause of inherited blindness (estimated prevalence 1:3000). Despite an intense effort to develop new treatments, our existing therapies to treat these retinal degenerations are extremely limited. The exact mechanism involved in the loss of the neurosensory retina is unknown, but there is increasing evidence that apoptosis of the photoreceptors and the retinal pigment epithelium (RPE) is a primary mechanism. The P23H rat model represents a common protein conformational disease found in humans. Age-related macular degeneration is likely to also represent a ‘multigenic’ protein conformational disease. The mechanism of cellular injury in both conditions is likely mediated through apoptosis. Epidemiologic prevalence data in the population of Wisconsin (quite similar to Minnesota) is well characterized for AMD (Beaver Dam Wisconsin) and could be readily compared based on a standardized grading system.

Bile Acids: Bile acids are essential for emulsifying lipids in the intestinal lumen, and their synthesis and transport drive bile formation and provide a degradation pathway for cholesterol. More recently, Steer et al. have demonstrated that UDCA (ursodeoxycholic acid) and TUDCA will interrupt apoptosis by blocking classic pathways, and induction of survival pathways, demonstrated both in vitro and in vivo. Specifically, TUDCA has been demonstrated to be neuroprotective in animal models of Huntington's disease, improved graft survival in Parkinsonian rats, and protect against neurologic injury after acute ischemic or hemorrhagic stroke (Low & Steer et al.). Preliminary work done with the rds mouse model of inherited retinal degeneration, demonstrated a dramatic protection of the inner nuclear layer of the retina in this inherited form of neurosensory retinal degeneration. Functional preservation of the electroretinographic response (functional test of vision) also demonstrated preservation of visual function in the mouse model.

Animal Studies: The P23H rat model of inherited retinal degeneration is an animal model of a common mutation found in >10% of autosomal dominantly inherited retinitis pigmentosa in humans. This animal model has been studied in the laboratory of Dr. Olsen at the University of Minnesota. The mechanism of retinal degeneration is mediated by apoptosis, but may follow a separate pathway than that of the rds mouse. The P23H rat model represents a protein conformational disorder that leads to retinal degeneration. Other examples of protein conformation disorders include Huntington's and Parkinson's disease.

The rds mouse degeneration is mediated largely through a mutation in the P-subunit of rod cGMP phoshodiesterease, leading to increased cGMP that is toxic to photoreceptors.

Preliminary Study: Homozygous line 1 and line 3 P23H rats (very strong model of rapid retinal degeneration, especially in the homozygous state), were given 100-200 mg/kg/d of TUDCA via subcutaneous injections while control animals will be given placebo vehicle only. Animals were sacrificed at intervals that correspond to the known retinal degenerations. Eyes were enucleated and the neurosensory retina was examined for signs of neuroprotection by counting the cell nuclei in the various retinal layers (FIG. 1; study design). For each animal studied, 14 sections were taken for each eye with 30-50 separate measurements performed and averaged for each section. Counting the average number of outer nuclear layer (ONL) cells was used to determine the level of retinal injury or loss.

A representative image of the retinal degeneration is shown in sequential histophathologic images representative of the time point based on days from birth, and the influence of TUDCA (FIG. 2). Note that by week 12 in the TUDCA treated line 1 animals, that there is a visible difference in the thickness of the drug treated ONL as compared to the vehicle. In the line 3 animal study, there is less noticeable difference between the drug treated and the control. (A Sprague Dawley animal with no retinal degeneration is used as the control slide for comparison to a normal healthy animal.)

Using line 1 animals (FIG. 3) there is no statistically significant differences at any time point after birth in animals treated the TUDCA as compared to vehicle controls (see week 7, 10, and 12). Additionally, animal weight recordings indicated a significant weight loss in treated animals as compared to controls by day 37 and beyond (p<0.05), suggesting systemic toxicity at these dosages.

Using line 3 animals (FIG. 4), we were able to demonstrate and trend toward a protective effect of TUDCA on the rate of retinal degeneration by counting the ONL layer thickness at the 9 week post-natal time point (p=0.16). However, this did not reach statistical significance. In the line 1 animals, there was no statistically significant protective effect (FIG. 4). Once again, animal weight recordings indicated a significant weight loss in treated animals as compared to controls by day 37 and beyond (p<0.05), suggesting systemic toxicity at these dosages.

Conclusions: This study indicates a trend toward protection in retinal degenerations in the P23H homozygous model. Using the P23H rat in a homozygous genetic state does not purely represent the human condition that is heterozygous. The less aggressive heterozygous model perhaps would perhaps be a better model to determine the effect of TUDCA on the degeneration of the P23H rat.

The complete disclosure of all patents, patent documents, and publications cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method for treating a visual disorder, the method comprising administering to a subject a compound selected from the group of a hydrophilic bile acid, salts thereof, analogs thereof, or combinations thereof.

2. The method of claim 1 wherein the hydrophilic bile acid is ursodeoxycholic acid.

3. The method of claim 1 wherein the compound administered is glycol- or tauro-ursodeoxycholic acid.

4. The method of claim 1 wherein the visual disorder is macular degeneration, retinitis pigmentosa, glaucoma, and/or retinal degeneration.

5. The method of claim 1 wherein administering to a subject comprises contacting the eye of the subject with a hydrophilic bile acid, salts thereof, analogs thereof, or combinations thereof.

6. The method of claim 1 wherein the compound is administered in combination with a pharmaceutically acceptable carrier.

7. The method of claim 1 wherein administering comprises administering parenterally.

8. The method of claim 1 wherein administering comprises administering the compound in eye drops.

9. A method for treating a visual disorder, the method comprising contacting the eye of a subject a compound selected from the group of a hydrophilic bile acid, salts thereof, analogs thereof, or combinations thereof, wherein the visual disorder is macular degeneration, retinitis pigmentosa, glaucoma, and/or retinal degeneration.

10. The method of claim 9 wherein the hydrophilic bile acid is ursodeoxycholic acid.

11. The method of claim 9 wherein the compound administered is glycol- or tauro-ursodeoxycholic acid.

12. The method of claim 9 wherein administering comprises administering the compound in eye drops.
